(19) United States
(12) Patent Application Publication
(10) Pub. No.: US 2007/0009553 A1
(43) Pub. Date: Jan. 11, 2007

(54) Bacterial strains of genus exiguobacterium, culture method and uses

(76) Inventors: Marie-Laure Fardeau, Les Pennes-Mirabeau (FR); Y. Combet-Blanc, Marseille (FR); Bernhard Ollivier, Roquevaire (FR)

Correspondence Address: NIXON & VANDERHYE, PC, 901 NORTH GLEBE ROAD, 11TH FLOOR, ARLINGTON, VA, 22203, US

(21) Appl. No.: 10/538,715

(22) PCT Filed: Dec. 10, 2003
(86) PCT No.: PCT/FR03/03665

§ 371 Date: Jun. 14, 2005

(30) Foreign Application Priority Data
Dec. 13, 2002 (FR) 02 15 865

(51) Int. Cl. A61K 39/02; C12N 1/20

(52) U.S. Cl. 424/234.1; 435/252.3

(57) ABSTRACT

The invention concerns bacterial strains of genus Exiguobacterium. Said strains are thermoresistant, capable of growing on sugar and starch substrates and/or capable of producing metabolites such as L(+) lactate. The invention is useful for producing metabolites such as L(+) lactate.

The invention relates to novel strains of the Exiguobacterium genus.

It also relates to a method for culturing these strains, and to industrial uses thereof.

The invention relates more particularly to bacterial strains as isolated from samples originating from deep-sea hydrothermal systems.

The study, by the inventors, of the samples taken has led them to isolate a novel species of Exiguobacterium that exhibits properties of great interest in various industrial fields.

The aim of the invention is therefore to provide strains of this novel species.

The invention is also directed toward providing protocols for culturing these strains, that specify the physicochemical conditions and the composition of the culture medium which make it possible to favorably produce cells and/or certain metabolites, more particularly L(+) lactate.

According to another aspect, the invention is directed toward the direct use of these strains, or that of their metabolites, in various industrial fields.

The bacterial strains of the invention are characterized in that they have a DNA sequence, at least part of which is capable of hybridizing with genomic or plasmid DNA of the strain deposited on Dec. 5, 2002, under the No. I-2962, with the Collection Nationale de Cultures de Microorganismes (C.N.C.M.) [French national collection of microorganism cultures], 25 rue du Docteur Roux, 75015 Paris.

Advantageously, at least 70% of the genome of the strains of the invention is capable of hybridizing with the DNA of the deposited strain.

The invention is directed in particular toward the bacterial strains defined above, characterized by the sequence SEQ ID No. 1 of the 16S rRNA:

GCGTGCCTAATACATGCAAGTCGAGCGCAGGAAGCCGTCTGAACCCTTCG
GGGGGACGACGGTGGAATGAGCGGCGGACGGGTGAGTAACACGTAAAGAA
CCTGCCCATAGGTCTGGGATAACCACGAGAAATCGGGGCTAATACCGGAT
GTGTCATCGGACCGCATGGTCCGCTGATGAAAGGCGCTCCGGCGTCGCCC
ATGGATGGCTTTGCGGTGCATTAGCTAGTTGGTGGGGTAACGGCCCACCA
AGGCGACGATGCATAGCCGACCTGAGAGGGTGATCGGCCACACTGGGACT
GAGACACGGCCCAGACTCCTACGGGAGGCAGCAGTAGGGAATCTTCCACA
ATGGACGAAAGTCTGATGGAGCAACGCCGCGTGAACGATGAAGGCTTTCG
GGTCGTAAAGTTCTGTTGTAAGGGAAGAACAAGTGCCGCAGGCAATGGCG
GCACCTTGACGGTACCTTGCGAGAAAGCCACGGCTAACTACGTGCCAGCA
GCCGCGGTAATACGTAGGTGGCAAGCGTTGTCCGGAATTATTGGGCGTAA
AGCGCGCGCAGGCGGCCTCTTAAGTCTGATGTGAAAGCCCCCGGCTCAAC
CGGGGAGGGCCATTGGAAACTGGGAGGCTTGAGTATAGGAGAGAAGAGTG
GAATTCCACGTGTAGCGGTGAAATGCGTAGAGATGTGGAGGAACACCAGT
GGCGAAGGCGACTCTTTGGCCTATAACTGACGCTGAGGCTGCGAAAGCGT
GGGGAGCAAACAGGATTAGATACCCTGGTAGTCCACGCCGTAAACGATGA
GTGCTAGGTGTTGGAGGGTTTCCGCCCTTCAGTGCTGAAGCTAACGCATT
AAGCACTCCGCCTGGGGAGTACGGTCGCAAGGCTGAAACTCAAAGGAATT
GACGGGGACCCGCACAAGCGGTGGAGCATGTGGTTTAATTCGAAGCAACG
CGAAGAACCTTACCAACTCTTGACATCCCCCTGACCGGTACAGAGATGTA
CCTTCCCCTTCGGGGGCAGGGGTGACAGGTGGTGCATGGTTGTCGTCAGC
TCGTGTCGTGAGATGTTGGGTTAAGTCCCGCAACGAGCGCAACCCTTGTC
CTTAGTTGCCAGCATTnAGTTGGGCACTCTAGGGAGACTGCCGGTGACAA
ACCGGAGGAAGGTGGGGATGACGTCAAATCATCATGCCCCTTATGAGTTG
GGCTACACACGTGCTACAATGGACGGTACAAAGGGCAGCGAAGCCGCGAG
GTGGAGCCAATCCCAGAAAGCCGTTCTCAGTTCGGATTGCAGGCTGCAAC
TCGCCTGCATGAAGTCGGAATCGCTAGTAATCGCAGGTCAGCATACTGCG
GTGAATACGTTCCCGGGTCTTGTACACACCGCCCGTCACACCACGAGAGT
TTGCAACACCCGAAGTCGGTGAGGTAACCGTAAGGAGCCAGCCGCCGAAG
GTGGGGCAGATGATTGGGGTGAAGTCGTAACAAGGTAGCCGTATCGGAAG
GTGCGGCTGA

or a sequence having more than 97% similarity with SEQ ID No. 1.

According to another aspect, these strains are characterized in that they are thermoresistant, saccharolytic and amylolytic and/or in that they are capable of producing lactate.

It will be noted that, advantageously, the lactate produced is more than 95% L(+) lactate.

The expression “thermoresistant” is intended to mean bacterial strains capable of growing at temperatures of the order of 40 to 50° C., at a pH of 5.4 to 9.15, with an optimum for growth at 45° C., at a pH of approximately 7.

The invention is more particularly directed toward strains of the Exiguobacterium genus as shown by comparison of the sequences of the 16S ribosomal RNA fraction.

These strains are also characterized in that they do not reduce sulfate, thiosulfate, sulfur or sulfite.

The bacterial strains of the invention are also characterized in that they are Gram-positive.

According to yet another provision, the guanine plus cytosine content of the DNA of the bacterial strains of the invention is of the order of 50 mol %.

The invention is in particular directed toward the bacterial strain deposited with the C.N.C.M. on Dec. 05, 2002, under the No. I-2962.

The identification reference of this strain is 10C. The taxonomic name used to denote it will be Exiguobacterium lactigenes sp. nov.

The mutants of the strains that correspond to the definitions above also fall within the scope of the invention provided that they conserve at least a 70% capacity for hybridization with the genomic DNA of the deposited strain.

In accordance with the invention, the bacterial strains defined above are obtained by culturing, under facultative anaerobic conditions, at a pH of 5.4 to 9.15, at 37° C., in a basic medium as defined below, containing a sugar that can be used as an energy source by these strains.

The bacterial strains of the invention are advantageously used in food fermentation processes. Their fermentative and enzymatic properties make it possible to advantageously replace therein and/or to supplement those attributed to the lactic acid bacteria normally used.

The ability of the strains of the invention to ferment a large variety of sugars, in particular D-glucose, D-fructose, D-galactose, D-mannose, mannitol, D-ribose, D-sucrose and DL-maltose, and starch constitutes a considerable asset. Some of these sugars (glucose, fructose, sucrose) that can potentially be used as energetic substrates are, in fact, available in large amount, in particular in fermentative sugar juices.

The possibility of acting on the metabolism of these strains by controlling the physicochemical parameters of the culture medium (pH, sugar/peptide ratio) broadens their field of application. Thus, it is possible, for example, to direct fermentation toward the production of cells and of cell metabolites, such as enzymes.

The invention is therefore also directed toward a method for producing metabolites, in particular L(+) lactate, characterized in that it comprises:
culturing a bacterial strain as defined above, under conditions suitable for its development and for the production of the desired metabolite,
recovering the metabolites produced, and then isolating the desired metabolite and purifying it.

The lactic acid produced by the strains of the invention is very advantageous since it consists of more than 95% L(+) lactate, which can be assimilated by higher organisms, whereas D(−) lactate is toxic in nature.

It is separated from the culture, and is concentrated, for example, by evaporation, where appropriate to dryness.

The concentrates or dry products are used as they are or are treated so as to form desired derivatives of lactic acid.

The uses of lactic acid or of esters thereof and other derivatives relate to many fields.

Lactic acid is thus used in the agrofoods industry, by incorporating it into drinks, beers, dairy products such as cream, cheese, butter or ice creams, or else jams.

It will advantageously be used in bread-making and in Viennese pastry-making as a surfactant, in the form, for example, of lactyl mono- and diglycerides and of sodium stearyl lactylate.

In the pharmaceutical industry, potassium lactate may constitute a substitute for sodium chloride that is particularly invaluable in cases of hypertension.

It is also used for its complexing properties, in particular complexing with iron and calcium, for treating deficiencies.

Finally, among the uses of lactic acid and of its salts and derivatives in the chemical industry, mention will be made of its use in the development of plastic resins, of adhesives, of pesticides or of textiles, or alternatively in paints, diluents and solvents, or for surface-treating metals.

The great advantage of lactic acid in polymer chemistry will be underlined, where it is used to produce polylactides and/or copolymers with, for example, polyalkylene oxides, polyvalent alcohols, glycolic acid, hydroxycarboxylic acids, copolymers of ethylene and of propylene, butyl rubbers or thermoplastic polyurethane elastomers. Various articles can be produced from these polymers and/or copolymers, in particular for packaging, films for medical uses for producing dressings, or else coating materials for surgical sutures.

Other characteristics and advantages of the invention are reported in the following description, given by way of example, which concerns strain 10C mentioned above, deposited with the C.N.C.M. under the No. I-2962.

a. Protocol for Isolating Strain 10C

The isolation was carried out using samples of deep-sea hydrothermal systems.

Culture Media and Methods

A basic medium containing (per liter of distilled water): 1 g of NH₄Cl, 0.3 g of KH₂PO₄, 0.3 g of K₂HPO₄, 25 g of NaCl, 0.2 g of CaCl₂, 0.1 g of KCl, 3 g of MgCl₂, 0.5 g of CH₃COONa, 0.5 g of cysteine-HCl, 0.1 g of yeast extract (Difco Laboratories), 10 ml of a Balch mineral solution (1) and 1 mg of resazurin, is used. The pH is adjusted to 7.3 with 10M KOH and the medium is brought to boiling under a stream of nitrogen and cooled to ambient temperature.

The compositions of the Balch mineral solution and of the Balch trace element solution are as follows:

Balch mineral solution

| | | |
|---|---|---|
| KH₂PO₄ | 6 | g |
| NH₄)₂SO₄ | 6 | g |
| NaCl | 12 | g |
| MgSO₄.7H₂O | 2.6 | g |
| CaCl₂.2H₂O | 0.16 | g |
| Distilled H₂O q.s. | 1000 | ml |

Balch trace element solution

| | | |
|---|---|---|
| Nitriloacetic acid | 1.5 | g |
| MnSO₄.2H₂O | 0.5 | g |
| MgSO₄.7H₂O | 3 | g |
| NaCl | 1 | g |
| FeSO₄.7H₂O | 0.1 | g |
| CoCl₂.6H₂O | 0.1 | g |
| CaCl₂.2H₂O | 0.1 | g |
| ZnCl₂ | 0.1 | g |
| CuSO₄.5H₂O | 0.01 | g |
| AlK(SO₄)₂ | 0.01 | g |
| H₃BO₃ | 0.01 | g |
| Na₂MoO₄ | 0.01 | g |
| Distilled H₂O q.s. | 1000 | ml |

The pH of the culture medium is adjusted to 7.3 with 10M KOH.

The medium is subsequently brought to boiling, and then cooled to ambient temperature and dispensed, under a stream of nitrogen, into Hungate tubes, at a rate of 5 ml per tube, and into serum flasks, at a rate of 20 ml, under a stream of nitrogen and carbon dioxide (80:20; v/v).

After treatment of the sealed containers in an autoclave at 110° C. for 45 min, Na₂S.9H₂O, Na₂CO₃ and glucose are added, from sterile solutions, which gives, respectively, concentrations of 0.04%, 0.2% and 20 mM.

In order to initiate the enrichment of the culture, a 20 ml sample of medium is inoculated and incubated at 37° C. The culture is purified using the Hungate roll tube method with a solidified medium containing 15 g/l of agar.

b. Description of the Strain

Strain 10C is a nonsporulant, facultative anaerobic, Gram-positive bacterium in rod form, with optimal growth at 45° C., at pH 7 and 0-2% of NaCl.

It is a heterotrophic strain which requires yeast extract in order to ferment sugars.

The temperature for growth of the strain is 12 to 50° C., at a pH of 5.4 to 9.1, and a concentration of NaCl of between 0 and 12%.

Optimal growth is observed at 45° C., at pH 7 and 0-2% of NaCl.

To promote growth, peptides, for example yeast extracts, will advantageously be added to a medium containing carbohydrates, in particular glucose, as an energy source.

Metabolic Properties

The fermentation of sugars produces essentially (L+) lactate (approximately 2 mol of lactate/mol of glucose fermented). Under suitable growth conditions, the production of formate, acetate and ethanol is observed.

Genetic Characteristics:

Strain 10C is characterized by a guanine +cytosine content in the DNA of 50.4 mol %.

The purification and extraction of the DNA, the amplification and the sequencing of the 16S rRNA were carried out according to (2), (3) and (4). The DNA was isolated by chromatography on hydroxyapatite according to the method of Cashion et al. (5). The DNA-PNA hybridization was carried out as described by De Ley et al. (6), with the modification described by Huss et al. (7) and Escara and Hutton (8), using a spectrophotometer model 2600 equipped with a 2527-R thermoprogrammer (Gilford Instrument Laboratories Inc., Oberlin, Ohio, USA).

The sequence of the 16S rRNA corresponds to SEQ ID No. 1 given above.

c. Table of differences in substrates between strain
10C and E. aurantiacum

| Substrates | 10C | Exig aurantiacum |
|---|---|---|
| Lactate | − | − |
| Benzoate | − | |
| Glucose | + | + |
| Fructose | + | − |
| Galactose | + | + |
| D-xylose | − | − |
| Mannose | + | − |
| Mannitol | + | + |
| Glycerol | − | + |
| Fumarate | − | + |
| Pyruvate | − | − |
| Arabinose | − | − |
| Ribose | + | + |
| Sorbose | − | − |
| Sucrose | + | + |
| Maltose | + | + |
| Acetate | − | − |
| Butyrate | − | − |
| Propionate | − | − |
| Casamino acids | − | − |
| Dulcitol | − | − |
| Lactose | − | − |
| Rhamnose | − | − |
| Melizitose | − | − |

d. Culture Methods and Uses

I—Production of Biomass by Fermentation of Sugar

The process is carried out in non-renewed medium.

The fermentation is regulated at a pH of 7 using an alkaline solution (sodium hydroxide, for example), and at a temperature of 45° C. A culture medium corresponding to the following composition is used:

| | |
|---|---|
| Glucose | to be calculated |
| Yeast extract/protein hydrolyzate | to be calculated |
| NH₄Cl | 1 g/l |
| NaCl | 0.5 g/l |
| KH₂PO₄ | 0.3 g/l |
| K₂HPO₄ | 0.3 g/l |
| MgCl₂.6H₂O | 0.2 g/l |
| KCl | 0.1 g/l |
| CaCl₂.2H₂O | 0.1 g/l |

The concentrations of sugar and of yeast extracts depend on the concentration of cells that it is desired to obtain.

The sugar is autoclaved separately from the rest of the culture medium, as are certain mineral salts that form a precipitate during autoclaving. They are subsequently added sterilely to the other part of the culture medium (yeast extract+minerals, autoclaved together), and the final volume is then adjusted with sterile distilled water. The example below makes it possible to understand more clearly the protocol for preparing the media:

Example of preparation of 16 liters of a medium containing 40 g/l of sucrose and 3 g/l of yeast extract:

1°) Weighing out and autoclaving

| | Concentration | Mass to be weighed out | |
|---|---|---|---|
| Sucrose | 40 g/l | 640 g | In approx. 500 ml |
| MgCl₂.6H₂O | 0.2 g/l | 3.2 g | of distilled water. |
| CaCl₂.2H₂O | 0.1 g/l | 1.6 g | Autoclaving 110° C., |
| | | | 20 to 30 min. |
| Yeast extract | 3 g/l | 48 g | In approx. 15 l of |
| NH₄Cl | 1 g/l | 16 g | distilled water. |
| KH₂PO₄ | 0.3 g/l | 4.8 g | Autoclaving 121° C. |
| K₂HPO₄ | 0.3 g/l | 4.8 g | 1 h 30. |
| NaCl | 0.5 g/l | 8 g | |
| KCl | 0.1 g/l | 1.6 g | |

NB:
the sugar is autoclaved in a small volume of liquid and for only 20 minutes at 110° C. in order to prevent hydrolysis of the sucrose. The magnesium and calcium salts are autoclaved separately from the other salts and from the yeast extract in order to prevent any precipitation.

2°) Assembly: the sugar-based solution is transferred into the 15 liters of medium containing the yeast extract, and then sterile distilled water is added to make the volume up to 16 liters. All these transfers are carried out sterilely, around the flame of a Bunsen burner, by means of a nitrogen overpressure applied in the vat to be emptied in order to propel the liquid.

3°) Homogenization: a stream of nitrogen N₂ is bubbled into the medium thus assembled in order to mix all the elements and to ensure anaerobiosis.

2. Fermentation Mode

The studies were carried out in batch mode, made continuous by linking batches together. This is a “feed-harvest”, or “repeat batch” system which can be represented diagrammatically by the sequential linking together of three steps: filling the fermenter with new medium, then batch-culturing the bacteria, then emptying out the fermentation must, leaving a tank starter for inoculation of the next batch, and again filling, etc.

From a practical point of view, the advantage of this system lies in the fact that the steps consisting in cleaning and sterilizing the fermenter between two batches are eliminated, and in the possibility of the process being automated. In fact, it is possible to program an automated device which triggers the emptying and filling operations according to the value of parameters acquired on line by means of regulating unit.

II. Production of Biomass by Fermentation of Starch

In other experiments, using a starch substrate and the buffered medium defined above (but with 10 g of starch per liter), a conversion of the starch giving more than 95% of L(+) lactate and traces of formate is obtained.

### BIBLIOGRAPHICAL REFERENCES

(1) Balch W. E. et al., 1979, Microbiol. Rev. 43, 260-296,
(2) Andrews K. T. & Patel B. K. C., 1996, Int. J. Syst. Bacteriol. 46, 265-269,
(3) Love C. A. et al., 1993, Syst. Appl. Microbiol. 16, 244-251,
(4) Redburn A. C. & Patel B. K. C., 1993, FEMS Microbiol. Lett. 113, 81-86.
(5) Cashion P., 1977, Anal. Biochem, 81:461-466.
(6) De Ley J, 1970, Eur. J. Biochem, 12:133-142,
(7) Huss V. A. R., 1983, J. Syst. Appl. Microbiol, 4: 184-192,
(8) Escara J. F., 1980, Biopolymers, 19: 1315-1327.

1. A bacterial strain, characterized in that it is Exiguobacterium of the lactigenes genus and in that it has a DNA sequence, at least part of which is capable of hybridizing with genomic or plasmid DNA of the strain deposited on Dec. 5, 2002, under the No. I-2962, with the Collection Nationale de Cultures de Microorganismes (C.N.C.M.) [French national collection of microorganism cultures).

2. The bacterial strain as claimed in claim 1, characterized in that at least 70% of its genome is capable of hybridizing with the DNA of the deposited strain.

3. The bacterial strain as claimed in claim 1, characterized by the sequence SEQ ID No. 1 of the 16S rRNA:
GCGTGCCTAATACATGCAAGTCGAGCGCAGGAAGCCGTCTGAACCCTTCG
GGGGGACGACGGTGGAATGAGCGGCGGACGGGTGAGTAACACGTAAAGAA
CCTGCCCATAGGTCTGGGATAACCACGAGAAATCGGGGCTAATACCGGAT
GTGTCATCGGACCGCATGGTCCGCTGATGAAAGGCGCTCCGGCGTCGCCC
ATGGATGGCTTTGCGGTGCATTAGCTAGTTGGTGGGGTAACGGCCCACCA
AGGCGACGATGCATAGCCGACCTGAGAGGGTGATCGGCCACACTGGGACT
GAGACACGGCCCAGACTCCTACGGGAGGCAGCAGTAGGGAATCTTCCACA
ATGGACGAAAGTCTGATGGAGCAACGCCGCGTGAACGATGAAGGCTTTCG
GGTCGTAAAGTTCTGTTGTAAGGGAAGAACAAGTGCCGCAGGCAATGGCG
GCACCTTGACGGTACCTTGCGAGAAAGCCACGGCTAACTACGTGCCAGCA
GCCGCGGTAATACGTAGGTGGCAAGCGTTGTCCGGAATTATTGGGCGTAA
AGCGCGCGCAGGCGGCCTCTTAAGTCTGATGTGAAAGCCCCCGGCTCAAC
CGGGGAGGGCCATTGGAAACTGGGAGGCTTGAGTATAGGAGAGAAGAGTG
GAATTCCACGTGTAGCGGTGAAATGCGTAGAGATGTGGAGGAACACCAGT
GGCGAAGGCGACTCTTTGGCCTATAACTGACGCTGAGGCTGCGAAAGCGT
GGGGAGCAAACAGGATTAGATACCCTGGTAGTCCACGCCGTAAACGATGA
GTGCTAGGTGTTGGAGGGTTTCCGCCCTTCAGTGCTGAAGCTAACGCATT
AAGCACTCCGCCTGGGGAGTACGGTCGCAAGGCTGAAACTCAAAGGAATT
GACGGGGACCCGCACAAGCGGTGGAGCATGTGGTTTAATTCGAAGCAACG
CGAAGAACCTTACCAACTCTTGACATCCCCCTGACCGGTACAGAGATGTA
CCTTCCCCTTCGGGGGCAGGGGTGACAGGTGGTGCATGGTTGTCGTCAGC
TCGTGTCGTGAGATGTTGGGTTAAGTCCCGCAACGAGCGCAACCCTTGTC
CTTAGTTGCCAGCATTnAGTTGGGCACTCTAGGGAGACTGCCGGTGACAA
ACCGGAGGAAGGTGGGGATGACGTCAAATCATCATGCCCCTTATGAGTTG
GGCTACACACGTGCTACAATGGACGGTACAAAGGGCAGCGAAGCCGCGAG
GTGGAGCCAATCCCAGAAAGCCGTTCTCAGTTCGGATTGCAGGCTGCAAC
TCGCCTGCATGAAGTCGGAATCGCTAGTAATCGCAGGTCAGCATACTGCG
GTGAATACGTTCCCGGGTCTTGTACACACCGCCCGTCACACCACGAGAGT
TTGCAACACCCGAAGTCGGTGAGGTAACCGTAAGGAGCCAGCCGCCGAAG
GTGGGGCAGATGATTGGGGTGAAGTCGTAACAAGGTAGCCGTATCGGAAG
GTGCGGCTGA
or a sequence having more than 97% similarity with SEQ ID No. 1.

4. The bacterial strain as claimed in claim 1, characterized in that it is thermoresistant, saccharolytic and amylolytic and/or capable of producing L(+) lactate.

5. The strain as claimed in claim 1 any one of claims 1, characterized by growth properties at temperatures of the order of 40 to 50° C., at a pH of 5.4 to 9.15, with an optimum for growth at 45° C., at a pH of approximately 7.

6. The bacterial strain as claimed in claim 1, characterized by a guanine plus cytosine content in its DNA of approximately 50 mol %.

7. A bacterial strain, characterized in that it is Exiguobacterium of the lactigenes genus and in that it has a DNA sequence, at least part of which is capable of hybridizing with genomic or plasmid DNA of the strain deposited on Dec. 5, 2002, under the No. I-2962, at the Collection Nationale de Cultures de Microorganismes (C.N.C.M.), these strains being thermoresistant, saccharolytic and amylolytic and/or capable of producing L(+) Lactate, having growth properties at temperatures of the order of 40 to 50° C., at a pH of 5.4 to 9.15, with an optimum for growth at 45° C., at a pH of approximately 7, and a guanine plus cytosine content in its DNA of approximately 50 mol %.

8. The bacterial strain deposited with the C.N.C.M. on Dec. 5, 2002, under the number I-2962.

9. A method for culturing the bacterial strain as claimed in claim 1, characterized in that the process is carried out under facultative anaerobic conditions, at a pH of approximately 5.4 to 9.15, at 37° C., in particular of 6.5 to 7.5, in a basic medium containing a sugar that can be used as an energy source by this strain.

10. The use of the bacterial strain as claimed in claim 1, in food fermentation processes.

11. A method for producing metabolites such as L(+) lactate, characterized in that it comprises:
culturing a bacterial strain as claimed in claim 1, under conditions suitable for its development and for the production of the desired metabolite,
recovering the metabolites produced, isolating the desired metabolite and purifying it.


SEQUENCE LISTING (1 sequence)

SEQ ID NO: 1 - DNA, 1510 bp, Artificial Sequence
  Description of Artificial Sequence Exiguobacterium acetylium
  misc_feature (1117)..(1117): unknown
gcgtgcctaatacatgcaagtcgagcgcaggaagccgtctgaacccttcggggggacgacggtggaatgagcggcggacgggtgagtaacacgtaaagaacctgcccataggtctgggataaccacgagaaatcggggctaataccggatgtgtcatcggaccgcatggtccgctgatgaaaggcgctccggcgtcgcccatggatggctttgcggtgcattagctagttggtggggtaacggcccaccaaggcgacgatgcatagccgacctgagagggtgatcggccacactgggactgagacacggcccagactcctacgggaggcagcagtagggaatcttccacaatggacgaaagtctgatggagcaacgccgcgtgaacgatgaaggctttcgggtcgtaaagttctgttgtaagggaagaacaagtgccgcaggcaatggcggcaccttgacggtaccttgcgagaaagccacggctaactacgtgccagcagccgcggtaatacgtaggtggcaagcgttgtccggaattattgggcgtaaagcgcgcgcaggcggcctcttaagtctgatgtgaaagcccccggctcaaccggggagggccattggaaactgggaggcttgagtataggagagaagagtggaattccacgtgtagcggtgaaatgcgtagagatgtggaggaacaccagtggcgaaggcgactctttggcctataactgacgctgaggctgcgaaagcgtggggagcaaacaggattagataccctggtagtccacgccgtaaacgatgagtgctaggtgttggagggtttccgcccttcagtgctgaagctaacgcattaagcactccgcctggggagtacggtcgcaaggctgaaactcaaaggaattgacggggacccgcacaagcggtggagcatgtggtttaattcgaagcaacgcgaagaaccttaccaactcttgacatccccctgaccggtacagagatgtaccttccccttcgggggcaggggtgacaggtggtgcatggttgtcgtcagctcgtgtcgtgagatgttgggttaagtcccgcaacgagcgcaacccttgtccttagttgccagcattnagttgggcactctagggagactgccggtgacaaaccggaggaaggtggggatgacgtcaaatcatcatgccccttatgagttgggctacacacgtgctacaatggacggtacaaagggcagcgaagccgcgaggtggagccaatcccagaaagccgttctcagttcggattgcaggctgcaactcgcctgcatgaagtcggaatcgctagtaatcgcaggtcagcatactgcggtgaatacgttcccgggtcttgtacacaccgcccgtcacaccacgagagtttgcaacacccgaagtcggtgaggtaaccgtaaggagccagccgccgaaggtggggcagatgattggggtgaagtcgtaacaaggtagccgtatcggaaggtgcggctga